(19) United States
(12) Patent Application Publication
(10) Pub. No.: US 2015/0359958 A1
(43) Pub. Date: Dec. 17, 2015

(54) METHOD FOR STERILIZATION OF A HOLLOW FIBER FILTER MODULE, HOLLOW FIBER FILTER MODULE COMPRISING A CLOSURE, AND OXYGEN ABSORBING CLOSURE

(71) Applicant: B. BRAUN AVITUM AG, MELSUNGEN (DE)

(72) Inventor: FALK KAESTNER, BRETNIG-HAUSWALDE (DE)

(21) Appl. No.: 14/735,786

(22) Filed: Jun. 10, 2015

(30) Foreign Application Priority Data
Jun. 17, 2014 (DE) 10 2014 108 530.2

(51) Int. Cl. A61M 1/34

(52) U.S. Cl. CPC A61M 1/34

(57) ABSTRACT

Methods of sterilizing a hollow fiber filter module, hollow fiber filter modules comprising closures and closures comprising an oxygen absorber are disclosed. A method according to the invention is distinguished in that the interior of the hollow fiber filter module is sealed off by means of the housing associated to the hollow fiber filter module and by means of the closures, and at least one of the closures comprises an oxygen absorber. A hollow fiber filter module according to the invention is characterized by the corresponding closures with or without an oxygen absorber.

### CROSS REFERENCE TO RELATED APPLICATIONS

This application claims priority to German application DE 10 2014 108 530.2 filed Jun. 17, 2014, the contents of such application being incorporated by reference herein.

### FIELD OF THE INVENTION

The present invention relates to a method for sterilization of a hollow fiber filter module.

In addition, the present invention relates to a hollow fiber filter module which can be used especially in dialysis apparatus for the external purification of blood. A closure, in particular a closure plug for hermetically closing a hollow fiber filter module, also pertains to the subject matter of the invention.

### BACKGROUND OF THE INVENTION

Hollow fiber filter modules or dialyzers of common current construction for use in dialysis apparatus for the external purification of blood essentially consist, on the one hand, of a hollow fiber bundle through which blood to be cleaned is conveyed, and on the other hand of a space which surrounds the individual hollow fibers of the hollow fiber bundle and through which the dialysis liquid is conveyed. The direction of flow of the dialysis liquid is opposite to the direction of flow of the blood in most cases. The hollow fibers or the walls of the hollow fibers consist of a semipermeable material which allows, on the one hand, the exchange of water and any substances dissolved therein and on the other hand prevents the exchange of blood cells.

Various methods are already known for sterilizing hollow fiber filter modules for their intended use in the medical field.

### DESCRIPTION OF THE RELATED ART

DE 27 16 178 A1, for instance, describes several methods in each of which a hollow fiber filter module to be sterilized is first packed up in a wrapping, the inner space of the wrapping is subsequently sterilized with a sterilization gas or by irradiation, then evacuated and finally refilled with a controlled gaseous atmosphere.

If in a method according to DE 27 16 178 A1 the inner space is sterilized gamma radiation, it has to be ensured in general that there is no oxygen in the vicinity of the hollow fibers, because said oxygen would interact or react with the material of the hollow fibers due to the gamma radiation and thus the membrane properties of the hollow fibers would deteriorate. Thus, in order to avoid such deterioration of the membrane properties during a hollow fiber filter module sterilization by irradiation, it has become habitual, as described in EP 0 759 873 B1, to collect the oxygen, which is enclosed together with the hollow fiber filter module in a wrapping, with an oxygen absorber and hence keep it away from the hollow fibers.

Various oxygen absorbers or corresponding substances and packagings are disclosed, for instance, in US 2001/0023232 A1, EP 0 898 508 B1, EP 0 664 824 B1 and EP 2 009 062 B1.

A problem with the usual implementation of the sterilization method according to EP 0 759 873 B1 is the fact that the gas-tight packaging requires an expensive welding process, that the volume enclosed by the packaging may vary greatly due to the resiliency of the packaging and therefore the capacity of the oxygen absorber has to be assessed to be unnecessarily large, and that the effort for a safe sterile packaging of a hollow fiber filter module, which packaging meets all transport and storage conditions, is quite considerable.

### SUMMARY OF THE INVENTION

Therefore, it is an object of the present invention to reduce the effort for the sterilization of a hollow fiber filter module, meeting the same or even higher safety-related requirements.

This object is achieved with respect to a sterilization method and with respect to a hollow fiber filter module and a hollow fiber filter closure by the features of the claims.

Advantageous further developments of the method according to aspects of the invention as well as of the devices according to aspects of the invention are also subject matter of the claims.

Hollow fiber filter modules or dialyzers which can be used in dialysis apparatus for the external purification of blood and are sterilized by a method according to aspects of the invention with gamma radiation, generically comprise a housing with at least two first ports leading to a first inner zone and at least two second ports leading to a second inner zone. The first ports are provided to introduce fresh dialysis liquid to the first inner zone of the hollow fiber filter module during blood purification and to discharge any used dialysis liquid, which may contain excess water coming from the blood to be cleaned. The second ports are provided to introduce uncleaned blood to the second inner zone and to discharge cleaned blood.

The sterilization method according to aspects of the invention is distinguished by the manner how the first and second inner zones are tightly closed, in particular in air-tight or hermetical fashion. In contrast to methods known hitherto, this is not realized with the aid of an additional packaging, but on the one hand with the housing and on the other hand with closures. In this context, the closures can be mounted to and dismounted from the corresponding ports of the housing without any tools and are connected to the corresponding ports in a frictional and/or form-fitting manner in the mounted state. By way of example, the ports are closed with closure plugs or closure caps which can be connected to the ports in frictional manner by a press fit or with a thread. It is also possible that the ports are closed with closures which can be connected to the ports in a form-fitting manner such as with a clip connection, or both in a form-fitting and frictional manner. Furthermore, for closing the ports at least once a closure is used which comprises an oxygen absorber with a reactive surface area through which the oxygen absorber receives or absorbs oxygen enclosed in the first and/or in the second inner zone. The oxygen absorber preferably comprises iron powder containing crystal water in order to implement the absorption process. Provided that the requirements with regard to medical applicability are met, other compositions are conceivable, too. The oxygen absorber allows to reduce the fraction of uncombined oxygen in the first and/or in second inner zone by a considerable amount, enabling a damage-free sterilization of the hollow fiber filter module by gamma radiation.

It is advantageous in the method according to aspects of the invention that the use of individual closures which can be mounted without any tool allows to save an additional packaging and thus a correspondingly required packaging device or packaging machine. Due to the fact that the housing of the hollow fiber filter module is used as a sterile barrier (and not a usually flexible packaging foil), it is not only possible to save the effort for ensuring the tightness and the storage and transport safety of the packaging foil, but also the capacity of the oxygen absorber can be correspondingly matched more precisely with the inner volume of the more stable housing of the hollow fiber filter module.

As the first and the second inner zones are separated from each other by a semipermeable membrane, the oxygen exchange between two inner zones is possible, but may need a longer time. It is therefore advantageous according to a further aspect of the invention if one port each of the first ports and of the second ports is closed by a closure comprising an oxygen absorber. Thus, it becomes not only unnecessary that the oxygen possibly has to flow or diffuse through the semipermeable membrane in order to reach the oxygen absorber, but it is also possible to adapt the capacities of the at least two oxygen absorbers to the respective volumes of the first and/or second inner zones.

According to a further aspect of the invention, the first and/or second inner zones can be flushed with a protective gas prior to closing the first and/or second inner zones. This is advantageous because this measure reduces the quantity of enclosed oxygen which has to be absorbed by the oxygen absorber(s).

Furthermore, it may be of advantage if the increase in volume of the oxygen absorber in the reaction with the oxygen is taken into consideration with respect to the reactive surface area of the oxygen absorber. An oxygen absorber according to aspects of the invention may therefore be distinguished in that the reactive surface area of the oxygen absorber is void of any undercuts which become overgrown by the increase in volume due to the reaction with absorbed oxygen in such a manner that parts of the undercut, which would in fact be able to receive oxygen, are isolated from the oxygen.

A hollow fiber filter module, according to aspects of the invention, which can be used in dialysis apparatus for the external purification of blood comprises a housing with at least two first ports leading to a first inner zone for supplying and discharging a dialysis liquid and with at least two second ports leading to a second inner zone for supplying and discharging blood.

A characterizing feature of a hollow fiber filter module according to aspects of the invention is that it comprises closures which each can be mounted to and dismounted from one of the ports of the housing without any tool and can be connected to the respective port in a frictional and/or form-fitting manner. The closures allow to tightly close the first and second inner zones with the housing, in particular in an air-tight or hermetical fashion, without the need of an additional packaging or wrapping. In order to reduce the quantity of enclosed uncombined oxygen in the first and/ or second inner zone and thus prevent detrimental reactions of the uncombined oxygen with the hollow fibers of the hollow fiber filter module during the sterilization by gamma radiation, at least one of the closures comprises an oxygen absorber comprising a reactive surface area with which any oxygen enclosed in the first and/or second inner zone can be received, collected or absorbed at least in part.

The closures can be mounted to and dismounted from the corresponding ports of the housing without any tools and are connected to the corresponding ports in a frictional and/or form-fitting manner in the mounted state. By way of example, the closures are implemented by closure plugs, closure caps or closure lids which can be connected to the ports in frictional manner by a press fit or with a thread. It is also possible to give the closures such a design that they can be connected to the ports in a form-fitting fashion such as with a clip connection, or both in a form-fitting and frictional manner.

The oxygen absorber preferably comprises iron powder containing crystal water in order to implement the absorption process. Provided that the requirements with regard to medical applicability are met, other compositions are conceivable as well.

Advantageously, each one of the closures for the first ports and of the closures for the second ports is provided with an oxygen absorber. Hence, the first and the second inner zones each may have a direct access to one of the oxygen absorbers, allowing to reduce the required waiting period up to the end of which the amount of absorbed oxygen is sufficiently large.

In order to ensure a safe and/or more ergonomic handling of the closures, it may be advantageous if the outer shape and/or appearance of the closures provided with an oxygen absorber differs from the outer shape and/or appearance of the closures without oxygen absorber. In this context, the term “outer shape and/or appearance” relates in particular to those parts of the closures which are accessible for persons from outside even if the closures are mounted to the hollow fiber filter module of the invention. Such different closures can then be distinguished in visual and/or haptic manner by the staff which is responsible for the hollow fiber filter module.

As set forth in the description of the method according to aspects of the invention, it may be advantageous if the increase in volume of the oxygen absorber in the reaction with the oxygen is taken into consideration with respect to the reactive surface area of the oxygen absorber. Thus, an oxygen absorber according to aspects of the invention may be distinguished in that the reactive surface area of the oxygen absorber is void of any undercuts which become overgrown by the increase in volume due to the reaction with absorbed oxygen in such a manner that parts of the undercut, which would in fact be able to receive oxygen, are isolated from the oxygen.

A closure according to aspects of the invention can be mounted to and dismounted from at least one port of a hollow fiber filter module, in particular a hollow fiber filter module according to aspects of the invention, without using a tool and can be connected to the respective port in frictional and/or form-fitting manner. The closure allows to tightly close at least one of the ports, in particular in air-tight or hermetical fashion.

A characteristic feature of the closure according to aspects of the invention is an oxygen absorber which is located in the closure and whose reactive surface area can absorb at least partially any uncombined oxygen which is enclosed in a first and/or second inner zone. Thus, the oxygen absorber is arranged in the closure in such a manner that the oxygen absorber, on the one hand, is in fluid connection with the first and/or second inner zone to be closed and, on the other hand, is sealed off by parts of the closure from the external atmosphere with respect to the first and/or second inner zones.

The oxygen absorber preferably comprises iron powder and crystal water.

Advantageously, the reactive surface area may be designed such that it has no undercuts which—after an increase in volume of the oxygen absorber due to the reaction with absorbed oxygen—would isolate parts of the reactive surface area from any oxygen in the first inner zone and/or in the second inner zone which is still not absorbed.

### BRIEF DESCRIPTION OF THE DRAWINGS

The invention is best understood from the following detailed description when read in connection with the accompanying drawings. Included in the drawings are the following figures:

FIG. 1 is a sectional view of a hollow fiber filter module which for carrying out a sterilization according to the prior art is enclosed in a packaging along with a separate oxygen absorber;

FIG. 2 is a sectional drawing of a hollow fiber filter module according to aspects of the invention;

FIG. 3a is a sectional view of a closure according to aspects of the invention comprising an oxygen absorber; and

FIG. 3b is a top view of the closure shown in FIG. 3a.

### DETAILED DESCRIPTION OF THE PREFERRED EMBODIMENTS

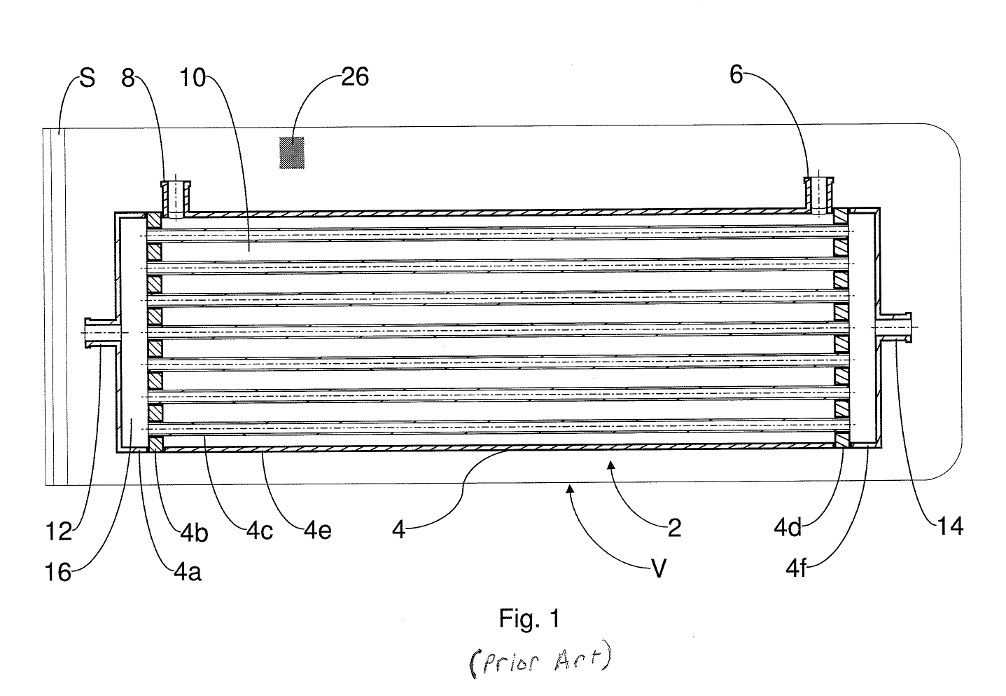

A generic hollow fiber filter module 2 as shown in FIG. 1 comprises a housing 4 which can be subdivided into a lid-shaped blood inlet section 4a, a hollow fiber bundle section 4b-d, an essentially cylindrical, in particular circularly cylindrical dialysis liquid section 4e and a likewise lid-shaped blood outlet section 4f.

The hollow fiber bundle section 4b-d in turn is formed from a first, in particular round hollow fiber bundle holder 4b embedding the ends of hollow fibers 4c, a hollow fiber bundle consisting of the hollow fibers 4c arranged parallel to one another, and a second, in particular round hollow fiber bundle holder 4d embedding the other ends of the hollow fibers 4c. Thus, the hollow fiber filter module 2 is sealed at the ends by potting, is mounted by the sealing compound and optionally cut at both ends after the sealing process in order to make any hollow fibers 4c again accessible which have been inadvertently closed by the sealing process.

A dialysis liquid section 4e of the housing 4, the former particularly having the shape of a circular cylinder shell, extends around the space of the hollow fiber bundle section 4b-d defined between the two hollow fiber bundle holders 4b and 4d. The space between the hollow fibers 4c, which is enclosed by the two hollow fiber bundle holders 4b and 4c and the dialysis liquid section 4e of the housing 4, corresponds to a first inner zone 10 of the hollow fiber filter module 2.

The lid-shaped blood inlet section 4a of the housing 4 is arranged or attached on the first hollow fiber bundle holder 4b on its side facing away from the hollow fiber bundle section 4e in such a manner that a blood distribution space can be defined between the blood inlet section 4a and the first hollow fiber bundle holder 4b.

The lid-shaped blood outlet section 4f of the housing 4 is arranged or attached on the second hollow fiber bundle holder 4d on its side facing away from the hollow fiber bundle section 4e in such a manner that a blood collection space can be defined between the blood outlet section 4f and the second hollow fiber bundle holder 4d.

The blood distribution space, the space within the hollow fibers 4c and the blood collection space in combination correspond to a second inner zone 16 of the hollow fiber filter module 2.

A port 12 for supplying blood is provided on the blood inlet section 4a of the housing 4, a port 14 for discharging blood is provided on the blood outlet section 4f of the housing 4 and, on the dialysis liquid section 4e of the housing 4, a dialysis liquid inlet 6 is provided in the vicinity of the second hollow fiber bundle holder 4d and a dialysis liquid outlet 8 is provided in the vicinity of the first hollow fiber bundle holder 4b.

As shown in FIG. 1, a generic hollow fiber filter module 2 according to the prior art is packed up—prior to a sterilization with gamma radiation—along with a separate oxygen absorber 26 in an especially bag-shaped packaging V made of plastics. In order to close the packaging V so as to be impermeable to gas, the open end is sealed by a weld seam S.

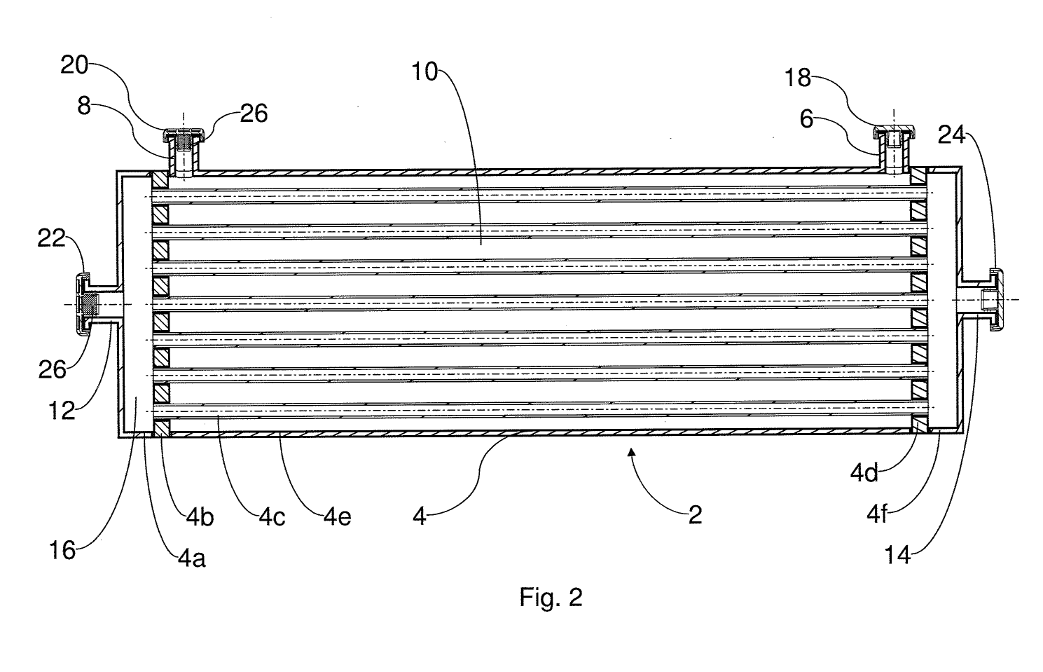

In comparison, FIG. 2 shows a sectional drawing, equivalent to FIG. 1, of a hollow fiber filter module according to aspects of the invention. In contrast to a conventional hollow fiber filter module, a hollow fiber filter module 2 according to aspects of the invention comprises closures 18, 20, 22 and 24 in the form of closure plugs which can be attached to the ports 6, 8, 12 and 14 without any tool in such a manner that the first inner zone 10 and the second inner zone 16 are closed so as to be impermeable to gas.

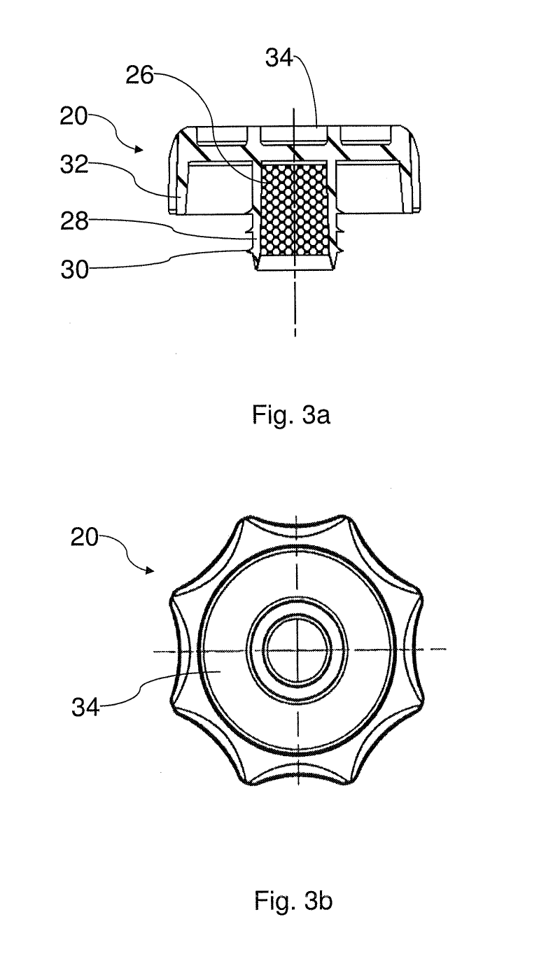

As is shown in the FIGS. 3a and 3b by the example of the closure 20, each of the closures 18, 20, 22 and 24 consists of a circularly cylindrically shaped closure piece 28 which has its outer wall provided with lamellas 30 in circumferential direction, and of a handling piece 32. Taking the closure 20 as an example, the sealing off of the corresponding port 8 having the shape of a circularly cylindrical shell is effected in that the closure 20 is grasped at its handling piece 32 and the circularly cylindrically shaped closure piece 28 of the closure 20 is moved to the port 8 such that the cylindrical closure piece 28 is inserted in the port 8 (having the shape of a cylinder shell) with a press fit. According to one embodiment, the press fit is established by the elastically deformable design of the entire closure 20. Embodiments in which either the lamellas 30 or only the closure piece 28 are/is realized so as to be elastically deformable, are also within the meaning of the invention. Any embodiments in which not the closures 18, 20, 22 and/or 24, but the corresponding ports 6, 8, 12 and/or 14 are designed to be completely or partially elastically deformable, are also within the scope of the invention.

The term “elastically deformable design” means here that the shape of the closure 20 and/or of the port 8 can be changed by a manual application of a compressive force in such a manner that the closure piece 20a can be inserted into the corresponding port 8 and that, having finished to exert a compressive force on the closure 20 and/or the port 8, the shape thereof is again restored in such a manner that the closure piece 28 or the cylindrical outer contour of the closure piece 28 at least partially rests against the port 8 or the cylindrical inner contour of the port 8 and closes the port 8 in a manner so as to be impermeable to gas. The elasticity can be implemented by a suitable selection of the material and/or by a suitable geometrical dimensioning.

The closure 20 comprises an oxygen absorber 26 on the closure piece 28, to be more precise on the side of the closure piece 28 which faces the corresponding inner zone 10 if the closure 20 is mounted on the hollow fiber filter module 2. The oxygen absorber 26 is realized by a porous structure containing iron powder and crystal water. The size of the oxygen absorber 26 is dimensioned to be proportional to the respective inner zones 10 and 16.

The closure piece 28 is an essentially round, sleeve-shaped shell which has a round interior in which the round, piston-shaped oxygen absorber 26 is arranged. The oxygen absorber 26 is frictionally connected to the closure piece 28 with a press fit; alternatively or in addition, it may also be cast, glued in situ or bonded by a form-fit connection. The outer cylinder surface of the closure piece 28 may cooperate with a corresponding inner cylinder surface of the port 8 and hence corresponds to the sealing surface. The circumferential lamellas 30 are provided to improve the sealing characteristics.

The round, sleeve-shaped closure piece 28 is designed to be open to one side such that the oxygen absorber 26 is in fluid connection with the surroundings of the closure 20. These surroundings correspond to the first or second inner zone 10 of a hollow fiber filter module 2 if the closure 20 is attached to the hollow fiber filter module 2. In addition, a membrane (not shown in the Figures) can be provided on the open side of the sleeve-shaped closure piece 28, ensuring on the one hand that any oxygen is able to reach the oxygen absorber 26, and on the other hand ensuring that parts which break off or separate from the oxygen absorber 26 do not leave the interior of the closure piece 28, preventing the potential risk of a contamination of the corresponding inner zone 10 of the hollow fiber filter module 2.

The other side of the round, sleeve-shaped closure piece 28 is concentrically adjoined by a plate-shaped part of the rotationally symmetrical handling piece 32. The plate-shaped part of the handling piece 32 is formed in one piece with the closure piece 28. Alternatively, the plate-shaped part of the handling piece 32 and the closure piece 28 can also have a multi-piece design. The plate-shaped part of the handling piece 32 adjoins the closure piece 28 in such a manner that the interior of the closure piece 28, in which the oxygen absorber 26 is arranged, corresponds to a blind hole.

A round, sleeve-shaped edge or an edge in the shape of a circularly cylindrical shell extends at the edge of the plate-shaped, rotationally symmetrical part of the handling piece 32 essentially parallel to the closure piece 28. The edge of the handling piece 32 has its circumference provided with recessed grips facilitating the gripping and handling of the closure 20. The edge of the handling piece 32 is arranged on the plate-shaped part of the handling piece 32 in such a manner that it encompasses a tubular part of the port 8 in part, similar to a crown cap, if the closure piece 28 is inserted in the tubular part of the port 8. Here, the inner surface of the edge of the handling piece 32 may rest against the outer surface of the tubular part of the port 8 and hence be part of the sealing surface. However, it is also possible that a ring-shaped gap remains between the inner surface of the edge of the handling piece 32 and the outer surface of the tubular part of the port 8. The inner surface of the edge of the handling piece 32 is formed to be slightly conical; alternatively, it may be circularly cylindrical as well.

As shown in FIG. 1, the ports 6, 8, 12 and 14 and hence the corresponding (not illustrated) closures 18, 20, 22 and 24 may each have the same size. According to the embodiment shown in FIG. 2, it is also possible that only the first ports 6 and 8 and the corresponding closures 18 and 20 or the second ports 12 and 14 and the corresponding closures 22 and 24 have the same size.

In order to be able to distinguish and generally identify the closures 20 and 22 provided with an oxygen absorber 26 from the equally large closures 18 and 24 without oxygen absorber 26 even in the mounted state, markings 34 in the form of ring-shaped depressions are provided on that side of the handling piece 32 of the closures 20 and 22 which faces away from the corresponding inner zone 10 or 16, if the closure 20 or 22 is mounted to the hollow fiber filter module 2. As an alternative to this, it would be possible to use any different reliefs and/or applied graphical information for improving the discriminability between the closures 20 and 22 comprising an oxygen absorber 26 and the equally large closures 18 and 24 without oxygen absorber.

The closures 20 and 22 are manufactured in the injection molding method from polypropylene. Alternatively, they may also be manufactured from polyoxymethylene, polyether ether ketone, polysulfone or from any other suitable plastics.

The embodiments of the hollow fiber filter module 2 according to aspects of the invention and of the closure 20 or 22 according to aspects of the invention which are shown in the FIGS. 2 to 3b and described above, only represent possible implementations of the claimed invention.

By way of example, the closures 18, 20, 22 and 24 may also be formed in a lid-shaped manner such that there is no closure piece 28 which is inserted in the corresponding port 6, 8, 12 or 14, but that the ports 6, 8, 12 or 14 are each received in a corresponding blind hole in the closure 18, 20, 22 or 24.

In order to connect the closures 18, 20, 22 and 24 in each case to the corresponding ports 6, 8, 12 and 14 not only by a frictional fit but also by an interlocking fit, the inner side of the cylinder shell-shaped ports 6, 8, 12 and 14 may be provided with depressions which are capable of receiving the lamellas 30.

1-10. (canceled)

11. A hollow fiber filter module for use with a gamma radiation-based sterilization method, the module including a housing having at least two first ports leading to a first inner zone for supplying and discharging a dialysis liquid and at least two second ports leading to a second inner zone for supplying and discharging blood, wherein:
the first and second inner zones are tightly closed in an air-tight or hermetic fashion by the housing and by closures, wherein each closure can be mounted to and dismantled from a respective port of the housing without using any tool and can be connected to the respective port in at least one of a frictional or a form-locking manner;
wherein at least one of the closures comprises an oxygen absorber having a reactive surface area absorbing at least partially any oxygen which is enclosed in at least one of the first inner zone or the second inner zone.

12. The module according to claim 11, wherein one of the first ports and one of the second ports is each closed by a respective closure comprising an oxygen absorber.

13. The module according to claim 11, wherein at least one of the first inner zone or the second inner zone is flushed with a protective gas prior to closing the at least one of the first inner zone or second inner zone.

14. The module according to claim 11, wherein the reactive surface area of the oxygen absorber is void of any undercuts which—after an increase in volume of the oxygen absorber due to the reaction with absorbed oxygen—would isolate parts of the reactive surface area from any oxygen which is enclosed in at least one of the first inner zone or the second inner zone and is still not absorbed.

15. A hollow fiber filter module which can be used in a dialysis apparatus for the external purification of blood, the module comprising:
a housing including at least two first ports leading to a first inner zone for supplying and discharging a dialysis liquid and at least two second ports leading to a second inner zone for supplying and discharging blood;
closures, wherein each closure can be mounted to and dismantled from a respective port of the housing without using any tool and can be connected to the respective port in at least one of a frictional or a form-locking manner;
wherein the first and the second inner zone can be closed tightly, in an air-tight or hermetic fashion, by the housing and the closures; and
wherein at least one of the closures comprises an oxygen absorber having a reactive surface area which is capable of absorbing any oxygen enclosed in at least one of the first inner zone or the second inner zone.

16. The hollow fiber filter module according to claim 15, wherein at least one of the closures for the first ports and at least one of the closures for the second ports is provided with an oxygen absorber.

17. The hollow fiber filter module according to claim 15, wherein the closures provided with the oxygen absorber differ from the closures without the oxygen absorber in at least one of their outer shape or their appearance in such a manner that they can be distinguished from outside in at least one of a visual or haptic manner when mounted to the hollow fiber filter module.

18. The hollow fiber filter module according to claim 17, wherein the closures for the first ports differ from the closures for the second ports in at least one of their outer shape or appearance in such a manner that they can be distinguished from outside in at least one of a visual or haptic manner when mounted to the hollow fiber filter module.

19. The hollow fiber filter module according to claim 15, wherein the closures for the first ports differ from the closures for the second ports in at least one of their outer shape or appearance in such a manner that they can be distinguished from outside in at least one of a visual or haptic manner when mounted to the hollow fiber filter module.

20. The hollow fiber filter module according to claim 15, wherein the reactive surface area of the oxygen absorber is void of any undercuts which—after an increase in volume of the oxygen absorber due to the reaction with absorbed oxygen—would isolate parts of the reactive surface area from any oxygen in at least one of the first inner zone or the second inner zone which is still not absorbed.

21. A closure for a hollow fiber filter module which can be mounted to and dismounted from at least one port of a hollow fiber filter module without using any tool and which can be connected to the at least one port in at least one of a frictional or form-locking manner to tightly close the at least one port in air-tight or hermetic fashion, wherein the closure comprises an oxygen absorber which has a reactive surface area to at least partially absorb oxygen enclosed in at least one of a first inner zone or a second inner zone.

22. The closure according to claim 21, wherein the reactive surface area of the oxygen absorber is void of any undercuts which—after an increase in volume of the oxygen absorber due to the reaction with absorbed oxygen—would isolate parts of the reactive surface area from any oxygen in at least one of the first inner zone or the second inner zone which is still not absorbed.
